SEQUENCE LISTING (2 sequences)

SEQ ID NO: 1 - DNA, 22 bp, Artificial Sequence
  Synthetic (hLINE1 Forward primer)
tcactcaaagccgctcaactac

SEQ ID NO: 2 - DNA, 24 bp, Artificial Sequence
  Synthetic (hLINE1 Reverse primer)
tctgccttcatttcgttatgtacc

(19) United States
(12) Patent Application Publication
(10) Pub. No.: US 2018/0327827 A1
(43) Pub. Date: Nov. 15, 2018

(54) METHOD OF ISOLATING NUCLEIC ACID

(71) Applicants: Samsung Electronics Co., Ltd., Suwon-si, Gyeonggi-do (KR); SAMSUNG LIFE PUBLIC WELFARE FOUNDATION, Seoul (KR)

(72) Inventors: Kyung Yeon Han, Seoul (KR); Dong Hyun Park, Gangwon-do (KR); Woongyang Park, Seoul (KR)

(21) Appl. No.: 15/756,944

(22) PCT Filed: Aug. 30, 2016
(86) PCT No.: PCT/KR2016/009660

§ 371 Date: Jul. 20, 2018

(30) Foreign Application Priority Data
Sep. 1, 2015 (KR) 10-2015-0123496

(51) Int. Cl. C12Q 1/6869; C12N 1/08; C12N 15/10; C12Q 1/6876; C12N 15/115; G01N 35/00

(52) U.S. Cl. CPC C12Q 1/6869; C12N 1/08; C12N 15/1006; C12Q 1/686; C12N 15/115; G01N 35/0098; C12Q 1/6876

(57) ABSTRACT

A method of isolating DNA and RNA from a single cell sample effectively is provided. By the method of isolating, it is possible to isolate DNA and RNA from a single cell sample, and thus genome information and transcriptome information can be simultaneously collected and/or analyzed.

### CROSS-REFERENCE TO RELATED APPLICATIONS

The present application claims priority to Korean Patent Application No. 10-2015-0123496 filed on Sep. 1, 2015, with the Korea Industrial Property Office, the entire disclosure of which is hereby incorporated by reference.

### BACKGROUND OF THE INVENTION

### 1. Field of the Invention

A method of isolating DNA and RNA in a cell sample is provided. By the method of isolating, it is possible to isolate DNA and RNA from a single cell, and thus genome information and transcriptome information can be simultaneously collected and/or analyzed.

### 2. Description of the Related Art

Recently, the importance of research on the analysis and interpretation of genetic information (e.g., genome or DNA, transcriptome or RNA) is enhanced in the fields of diagnosis of diseases, establishment of therapeutic strategy, and treatment monitoring.

For such an analysis of genetic information, it is important to effectively isolate a nucleic acid like DNA or RNA from a sample.

In this regard, U.S. Pat. No. 5,777,098 provides a method of isolating/purifying intracellular DNA, but does not disclose isolation and analysis of RNA, and thus there is an inconvenience of performing a treatment process for isolation and purification of RNA from another sample separately.

Thus, the development of a method of isolating a nucleic acid such as DNA or RNA more simply and effectively is required.

### SUMMARY OF THE INVENTION

One embodiment of the present invention provides a method of isolating a nucleic acid, which can simultaneously isolate (separate) DNA and RNA from a single cell.

More specifically, the method of isolating a nucleic acid may comprise,

(1) preparing a cell sample comprising a target cell (for example, a cell sample comprising a single target cell);

(2) treating the cell sample with a bead, to which a targeting material binding to a protein on the membrane of the target cell is attached, to bind the bead to the membrane of the target cell;

(3) obtaining a cell lysate by dissolving the cell membrane by treating a hypotonic solution to the cell sample;

(4) obtaining a liquid portion and a solid portion of the obtained cell lysate;

(5) isolating RNA from the liquid portion obtained from the step (4); and

(6) isolating DNA from the solid portion obtained from the step (4).

### BRIEF DESCRIPTION OF THE DRAWINGS

FIG. 1 is a schematic diagram which shows selective lysis of the cell membrane using hypotonic lysis.

FIG. 2 is a schematic diagram which shows a process of selective isolation of RNA and DNA.

FIG. 3 is a fluorescence image obtained after the treatment of the isotonic solution (PBS; pH 7.4) and the hypotonic solution (⅕ PBS) to the cell stained by cytoplasm staining (CellTracker, green) and nucleus staining (DAPI, blue), and shows that the cell membrane is selectively lysed by the hypotonic solution.

FIG. 4 is a graph of comparing the result of quantification of DNA isolated according to the method of Example 1 with the result obtained from the whole cell lysate.

FIG. 5 is a graph of comparing the result of quantification of RNA isolated according to the method of Example 1 with the result obtained from the whole cell lysate.

FIG. 6 is a graph of comparing the recovery rate of RNA isolated according to the method of Example 1 with the result obtained from the whole cell lysate.

FIG. 7 is a graph of comparing the recovery rate of DNA isolated according to the method of Example 1 with the result obtained from the whole cell lysate.

FIG. 8 shows the results of analysis of correlation between sequences of MCF7 bulk sample RNA, whole cell RNA, and fractionated RNA.

FIG. 9 is a graph which shows the result of RNA sequencing of fractionated RNA (detected gene number).

FIG. 10 is a graph which shows the result of sequencing of the whole length of genome to DNA fraction isolated according to the method of Example 1 by comparing with the result obtained from the bulk sample and whole cell lysate.

### DETAILED DESCRIPTION

A method for simultaneously isolating DNA and RNA from cell samples is provided.

In the present specification, beads are bound to the cell membrane of a target cell by using beads in which a targeting material that binds to a protein positioned in the cell surface (external exposed portion of cell membrane) of the target cell in a cell sample (e.g., antibody, polypeptide, aptamer,) is attached (connected). The cell is lysed by using a hypotonic solution. The cell membrane of lysed target cell is present in cell lysates in a state bound to beads, and RNA is present supernatants in a state released from cell lysates.

When the concentration of the hypotonic solution is controlled, only the cell membrane is dissolved and the nuclear membrane is maintained, and thereby the complete state of nucleus is present in cell lysates. Since maintained nuclear membrane is connected to the cell membrane by cytoskeleton proteins such as microfilament (e.g., actin) and microtubule (e.g., tubulin), and the like, the integrity of the nucleus remains relatively well-preserved.

When the cell lysates are physically separated, a supernatant comprising RNA isolated (eluted) from the cell and the cell membrane bound to beads and nucleus are obtained. RNA can be isolated from the obtained supernatant, and DNA present in the nucleus can be isolated from the obtained precipitate.

The present invention, is completed through research as above and provides a method of isolating a nucleic acid, which simultaneously isolates DNA and RNA from the same cell sample.

The method of isolating a nucleic acid may comprise,

(1) preparing a cell sample comprising a target cell;

(2) treating the cell sample with a bead, to which a targeting material binding to a protein on the membrane of the target cell is attached, to bind the bead to the membrane of the target cell;

(3) obtaining a cell lysate by dissolving the cell membrane by treating a hypotonic solution to the cell sample;

(4) obtaining a liquid portion and a solid portion of the obtained cell lysate;

(5) isolating RNA from the liquid portion obtained from the step (4); and

(6) isolating DNA from the solid portion obtained from the step (4).

In case that the cell sample is a bulk sample comprising large number of cells, a step of single cell sampling, which samples one target cell, is required. Thus, in case that the cell sample is the bulk sample comprising large number of cells, the method of isolating a nucleic acid, between the step (2) and step (3), may further comprise step (2-1), a step of single cell sampling, which extracts one target cell bound to beads from the reacted material. The step (2-1) single cell extracting step may comprise a step of isolating one target cell bound to beads and aliquoting it on a reactor (for example, tube, well plate, etc.). In one embodiment, the isolation of one target cell, may be conducted by, for example, FACS (Fluorescence Activated Cell Sorting) method, but not limited thereto, and may be conducted by common cell isolation methods.

In the step (1), the target cell means a cell requiring isolation and/or analysis of nucleic acid information. The cell sample comprises cells isolated in vivo, and may comprise only the target cell, or comprise various kinds together with the target cell, or comprise the cell with buffer such as PBS or medium. The target cell may be all of cells in which the marker binding molecule (targeting material) can bind to surface-attached beads, as intrinsic surface markers (e.g., EpCAM) are known. The target cell and/or cell comprised in the cell sample may be selected from all of eukaryotes, and for example, it may be at least one selected from the group consisting of an animal cell, a plant cell, a bacterium, a fungus. The cell may be the cell derived from an animal, a plant, a bacterium, a fungus, and/or the culture of the cell. The cell may be all types of cells or cell lines such as a somatic cell, a germ cell, a stem cell like embryonic stem cell, adult stem cell, induced pluripotent stem cell, mesenchymal stem cell, etc, a gene modified cell and the like. The cell may be a normal cell and/or tumor cell/cancer cell (e.g., cancer cell in tissue or blood, intraperitoneal cancer cell), an inflammatory cell, an abnormal cell like chromosomal abnormal cell. The cell sample may be a cell obtained (isolated) from a patient, cell line, or culture thereof and the patient may be mammal including human.

When various kinds or large number of cells are comprised in the cell sample (large population), constructed genome and transcriptome information and the conventional sequencing performed on that basis may not represent the dynamic property of each individual cell and heterogeneity of individual cells. In addition, in this case, since DNA and RNA are obtained in the form of DNA mixture and RNA mixture derived from large number of cells, it is very difficult to match DNA and RNA by their original cell. For the accurate analysis of modification of genome and/or transcriptome covered with bulk signal and the accurate matching of genome (DNA) and transcriptome (RNAs) derived from individual target cell, it may be advantageous to conduct analysis in the level of single cell.

Thus, in one embodiment, the target cell may be a single cell, and the cell sample may be a single cell sample comprising a single target cell. When the cell sample comprises a number of cells, as aforementioned, the step (2-1) single cell extracting step may be additionally conducted.

However, there are problems in obtaining a sample, reverse transcription and cDNA synthesis step, because RNA is present in an extremely small amounts in the single cell sample. These experimental difficulties make accurate analysis of biological variation difficult.

The method of isolating a nucleic acid provided in the present invention, has an advantage that can represent dynamic properties and heterogeneity of an individual cells, and simultaneously conduct accurate analysis with a very small amount of RNA, since it can provide a cDNA library available for the whole transcriptome analysis by effectively extracting sub-pg levels of RNA from a single cell with very little loss. In addition, there is an advantage that isolation is possible without separate tagging and/or pretreatment when extracting RNA.

In the step (2), the bead is not particularly limited as long as it is a solid material, and it may be at least one selected from the group consisting of a magnetic bead, a silica bead, a polymer bead (for example, polystyrene bead, etc.), a glass bead, a cellulose bead, a quantum dot (Q-dot), a metal bead (for example, silver (Au), gold (Ag), copper (Cu), etc.) and combinations thereof. For example, the bead may be a magnetic bead. The magnetic bead is a core/shell structure in which a magnetic particle and the external surface of the magnetic particle are coated with silica, metal, polymer, and in this case, there is an advantage that unreacted cells can be easily removed by using a magnet after the reaction with the cell sample and a liquid portion and a solid portion can be easily isolated by using the magnet without centrifugation after cell lysis in the following step. In addition, the magnetic bead has an advantage that can easily isolate the product without a loss from a trace amount of sample in the microgram level at the time of single cell targeting.

The size of bead is not particularly limited, but when the diameter of bead is too small, isolation without bead aggregation is difficult, and when it is too large, cell may be damaged during cell-bead connection reaction, and therefore it is advantageous to control it in an appropriate size. For example, the bead, for effective cell membrane protein attachment and isolation, may have an average diameter of 1 μm to 20 μm, 1 μm to 15 μm, 1 μm to 10 μm, 5 μm to 20 μm, 5 μm to 15 μm, 5 μm to 10 μm, 10 μm to 20 μm, or 10 μm to 15 μm. In addition, the bead may be a mixture of beads having two or more sizes. In other words, the bead may be of the same size or a mixture of beads having different sizes each other.

The targeting material attached on the surface of the bead may be at least one selected from the group consisting of an antibody specifically combinable to a protein present in the cell membrane of the target cell, an antigen binding fragment of the antibody, a protein scaffold like DARPin, aptamer, a small molecule compound, and the like. The targeting material may be properly selected according to the kind of target cell.

The protein present in the cell membrane of the target cell, may be, for example, all proteins which are exposed to the external (extracellular) surface of the cell membrane in whole or part, and for example, may be selected form the group consisting of various kinds of receptors, transmembrane glycoprotein (for example, epithelial cell adhesion molecule (EpCAM), etc.), and the like. The receptor may be a receptor tyrosine kinase protein, and for example, may be selected from the group consisting of various kinds of growth factor (for example, EGF (Epidermal growth factor), PDGF (Platelet-derived growth factor), FGF (fibroblast growth factor), VEGF (vascular endothelial growth factor), etc.) receptors. The receptor may be selected from the group consisting of for example, ErbB family including EGFR (Epidermal growth factor receptor), HER2, HER3, insulin receptor, PDGF receptor (Platelet-derived growth factor receptor; PDGFR), FGF receptor (fibroblast growth factor receptor; FGFR), VEGF receptor (vascular endothelial growth factor receptor; VEGFR), HGF receptor including c-Met, etc. (hepatocyte growth factor receptor; HGFR), Trk receptor (tropomyosin-receptor-kinase receptor), Eph receptor (Ephrin receptor), AXL receptor, LTK receptor (Leukocyte receptor tyrosine kinase), TIE receptor, ROR receptor (receptor tyrosine kinase-like orphan receptor), DDR receptor (Discoidin domain receptor), RET receptor, KLG receptor, RYK receptor (related to receptor tyrosine kinase receptor), MuSK receptor (Muscle-Specific Kinase receptor). In one embodiment, the protein present in the cell membrane of the target cell may be a tumor cell/cancer cell surface specific marker protein.

The antibody may be antibody of all subtypes which recognizes the protein present in the cell membrane of the target cell as an antigen (IgA, IgD, IgE, IgG (IgG1, IgG2, IgG3, IgG4,), or IgM). The antigen binding fragment means a polypeptide comprising a portion specifically binding to the antigen, namely the protein present in the cell membrane of the target cell, and means heavy chain CDR (complementarity determining region), light chain CDR, heavy chain variable region, light chain variable region of antibody or combinations thereof (for example, scFv, (scFv)2, scFv-Fc, Fab, Fab′ or F(ab′)2).

The protein scaffold is a protein structure having a similar structure to the protein or having properties of specifically binding (and/or recognizing) to the specific protein or the specific cell, and for example, may be at least one selected from the group consisting of DARPin, Affibody, Lasso, Cyclotide, Knottin, Avimer, Kunitz Domain, Anticalin, Adnectin, Pronectin, Fynomer, Nanofitin, Affilin, but not limited thereto.

The targeting material may be attached to the bead surface by, for example, an ionic bond, a covalent bond, a non-covalent bond like adsorption, a ligand-receptor bond. For example, the bead may have the surface combinable to the targeting material itself, or may have the surface in which a functional group combinable to the targeting material is coated (surface modified).

The functional group which is possible to be coated on the bead surface, for example, may be at least one selected from the group consisting of amine-based compound wherein amine coupling (NH₂ coupling) is possible, thiol-based compound wherein thiol coupling (SH coupling) is possible, carboxyl-based compound wherein carboxyl coupling (COO coupling) is possible, antibody binding protein such as protein G, protein A, and the like, but not limited thereto, and it may be appropriately selected according to the kind of targeting material. For example, the functional group may be at least one selected from the group consisting of maleimide-based compound, pyridyldithio-based compound, N-hydroxysuccinimide-based compound, aldehyde, protein G, protein A, but not limited thereto.

In another embodiment, the targeting material may bind to the bead surface by a ligand-receptor bond like streptavidin-biotin bond. In other words, it is possible to attach the targeting material to the bead surface by the ligand-receptor bond, by attaching one of ligand and receptor to the bead surface and attaching the other to the targeting material. For example, when streptavidin is attached on the bead surface and biotin is bound to the targeting material with the common method and they react, the targeting material is attached on the bead surface by the interaction between strepavidin of the bead surface and biotin attached to the targeting material.

The bead in which the targeting material is attached of the step (2) may be prepared and used, or used by acquiring commercially available products. When prepared and used, a step of attaching the targeting material to the bead surface may be further comprised prior to the step (2). The step of attaching the targeting material to the bead surface may be reacted for the time enough for the targeting material to be applied (added or contacted) to the bead and to be bound on the bead surface at, 0 to 35 □ or 10 to 30□, for example, a room temperature, for example, 0.5 to 24 hrs, 0.5 to 12 hrs, 0.5 to 6 hrs, 1 to 24 hrs, 1 to 12 hrs, or 1 to 5 hrs, but not limited thereto, and it may be properly controlled with regard to kinds of bead and targeting material, etc. The amount of targeting material to be applied for attachment to the bead surface may be properly controlled according to the kinds of bead and/or targeting material, and for example, the maximum capacity combinable to the bead surface (namely, saturated capacity) (for example, in case of using an antibody as the targeting material, the maximum capacity (saturated capacity) combinable to the bead surface, obtained through titration of antibody amount combinable to the bead surface) or over-capacity exceeding it may be reacted, but not limited thereto.

The process of treating the bead of the step (2) to the cell sample may be carried out by adding the bead in which the targeting material is attached to the cell sample. When the number of added beads is too large, it may disturb the latter part of molecule analysis step, and when it is too small, the cell is not effectively attached, and therefore, the number of added beads may be controlled in the appropriate range. For example, the number of added beads may be 1 to 100 times, 1 to 50 times, 1 to 20 times, 1 to 15 times, 5 to 100 times, 5 to 50 times, 5 to 20 times, 5 to 15 times, 7 to 100 times, 7 to 50 times, 7 to 20 times, or 7 to 15 times of the number of cells in the cell sample, but not limited thereto, and it may be properly controlled with regard to kinds of target cell, kinds of targeting materials attached to the bead surface.

In order to bind the targeting material of the bead surface to the surface protein of the target cell, they may be reacted at 0 to 35 □ or 10 to 30□, for example, at room temperature, for 1 to 60 min, 5 to 30 min, or 10 to 20 min, after treating beads to the cell sample in the step (2), but not limited thereto, and it may be properly controlled with regard to kinds of target cell, kinds of targeting materials attached to the bead surface.

In case of using a magnetic bead, a step of removing unreacted (unbound) cells by applying a magnetic field generator like magnetic and washing reacted materials may be further comprised after the step (2) (for example, between the step (2) and the step (3)).

In the step (3), the hypotonic solution may be a buffer solution, or a surfactant solution in which a surfactant is dissolved in water or a buffer solution. The composition of the hypotonic solution may be properly controlled according to the DNA/RNA isolation efficiency.

The buffer may be selected from all biocompatible buffers, and but not limited thereto, with regard to biocompatibility, those having pH 7.2 to 7.6, for example, pH 7.4 may be used. For example, the buffer may be at least one selected from the group consisting of phosphate buffer saline (PBS), Hank's balanced saline solution (HBSS), and for example, may be PBS.

The surfactant may be at least one selected from the group consisting of cationic surfactant, anionic surfactant, non-ionic surfactant, or ampholytic surfactant. The cationic surfactant may comprise dodecyl trimethyl ammonium bromide, dodecyl trimethyl ammonium chloride, cetyl trimethyl ammonium bromide, and the anionic surfactant may comprise sodium dodecyl sulfate (SDS), sodium cholic acid, sodium dodecyl cholic acid, sodium N-lauroyl sarcosinate, and the non-ionic surfactant may comprise polyoxyethylene octylphenylether (for example, Triton X-100), polysorbate (for example, polyoxyethylenesorbitanmonolaurate (Tween20), polyoxyethylenesorbitanmonooleate (Tween80)), n-octyl-β-D-glucoside, n-octyl-β-D-glucopyranoside, n-octyl thio-β-D-thio glucopyranoside, octyl phenyl-ethoxy ethanol (for example, Nonidet P-40 (NP40)), polyethyleme-lauryl ester (for example, Brij35), polyethylene-glycol hexadecyl-ester (for example, Brij58), and the ampholytic surfactant may comprise 3-[(3-Cholamidopropyl)dimethylammonio]-1-propanesulfonate (CHAPS), phosphatidylethanolamine. In a specific embodiment, with regard to the effect on the nucleic acid, the surfactant may be at least one selected from the group consisting of polyoxyethylene octylphenylether (for example, Triton X-100), polysorbate (for example, polyoxyethylenesorbitanmonolaurate (Tween20), polyoxyethylenesorbitanmonooleate (Tween80)), and 3-[(3-Cholamidopropyl)dimethylammonio]-1-propanesulfonate.

The cell lysate obtained in the step (3) is characterized in that the nucleus is present in a complete state, as the cell membrane is lysed (destroyed) but the nuclear membrane is maintained. When the concentration of the hypotonic solution used in step (3) is too high, the lysis of cell membrane does not occur, and when it is too low, the nuclear membrane as well as the cell membrane is lysed. Thus, the hypotonic solution is characterized by having the concentration in the range of lysing the cell membrane and maintaining the nuclear membrane. For this, the mixing ratio of water and buffer of the buffer aqueous solution (water volume:buffer volume; total 100) may be 95:5 to 60:40, 95:5 to 70:30, 95:5 to 75:25, 95:5 to 78:22, 95:5 to 80:20, 90:10 to 60:40, 90:10 to 70:30, 90:10 to 75:25, 90:10 to 78:22, 90:10 to 80:20, 85:15 to 60:40, 85:15 to 70:30, 85:15 to 75:25, 85:15 to 78:22, 85:15 to 80:20, 82:18 to 60:40, 82:18 to 70:30, 82:18 to 75:25, or 82:18 to 78:22 by volume. In a specific embodiment, the buffer aqueous solution may be PBS solution in which water and PBS are mixed in the volume ratio (water volume:buffer volume; total 100) of 95:5 to 60:40, 95:5 to 70:30, 95:5 to 75:25, 95:5 to 78:22, 95:5 to 80:20, 90:10 to 60:40, 90:10 to 70:30, 90:10 to 75:25, 90:10 to 78:22, 90:10 to 80:20, 85:15 to 60:40, 85:15 to 70:30, 85:15 to 75:25, 85:15 to 78:22, 85:15 to 80:20, 82:18 to 60:40, 82:18 to 70:30, 82:18 to 75:25, or 82:18 to 78:22. In addition, the concentration of the surfactant to the water or buffer aqueous solution (namely, the volume of surfactant contained, when the volume of water or buffer aqueous solution is 100) may be 0.01 to 10% (v/v), 0.01 to 5% (v/v), 0.01 to 1% (v/v), 0.01 to 0.5% (v/v), 0.01 to 0.3% (v/v), 0.05 to 10% (v/v), 0.05 to 5% (v/v), 0.05 to 1% (v/v), 0.05 to 0.5% (v/v), 0.05 to 0.3% (v/v), 0.08 to 10% (v/v), 0.08 to 5% (v/v), 0.08 to 1% (v/v), 0.08 to 0.5% (v/v), or 0.08 to 0.3% (v/v). In addition, the mixing ratio of water and buffer in the buffer aqueous solution used as a solvent (water volume:buffer volume) may be 95:5 to 60:40, 95:5 to 70:30, 95:5 to 75:25, 95:5 to 78:22, 95:5 to 80:20, 90:10 to 60:40, 90:10 to 70:30, 90:10 to 75:25, 90:10 to 78:22, 90:10 to 80:20, 85:15 to 60:40, 85:15 to 70:30, 85:15 to 75:25, 85:15 to 78:22, 85:15 to 80:20, 82:18 to 60:40, 82:18 to 70:30, 82:18 to 75:25, or 82:18 to 78:22. In one embodiment, in order to prevent decomposition of RNA eluted by cell membrane lysis, prior to, after, or at the same time of treatment of the hypotonic solution, RNase inhibitor may be additionally treated. The kind of RNase inhibitor is not particularly limited, and it may be properly selected from all types of RNase (for example, RNase A, B, C) inhibitors used commonly. When the RNase inhibitor is treated together by being comprised in the hypotonic solution, the content of RNase inhibitor in the hypotonic solution may be 0 to 10% (v/v), 0 to 5% (v/v), 0 to 2% (v/v), 0.1 to 10% (v/v), 0.1 to 5% (v/v), or 0.1 to 2% (v/v), but not limited thereto, and it may be properly adjusted according to the kind of RNase inhibitor. In addition, the amount used of the hypotonic solution may be used as 5 to 20 μl, 5 to 15 μl, 5 to 10 μl, 8 to 20 μl, 8 to 15 μl, or 8 to 10 μl based on the cell solution 1 μl, for example, 1:9 based on the volume as the ratio of cell solution:hypotonic solution, but not limited thereto. The cell solution is a surfactant solution comprising a single target cell in which the bead is bound, and the surfactant is as aforementioned.

In step (3), in order to achieve proper cell lysis, after treating the hypotonic solution to the cell sample in the step (3), it may be reacted for example, at 0 to 35 □ or 10 to 30 □, for example, at room temperature, for 1 to 60 min, 3 to 30 min, or 5 to 20 min, but not limited thereto, and it may be properly controlled with regard to the kind of target cell, the kind and/or concentration of used hypotonic solution.

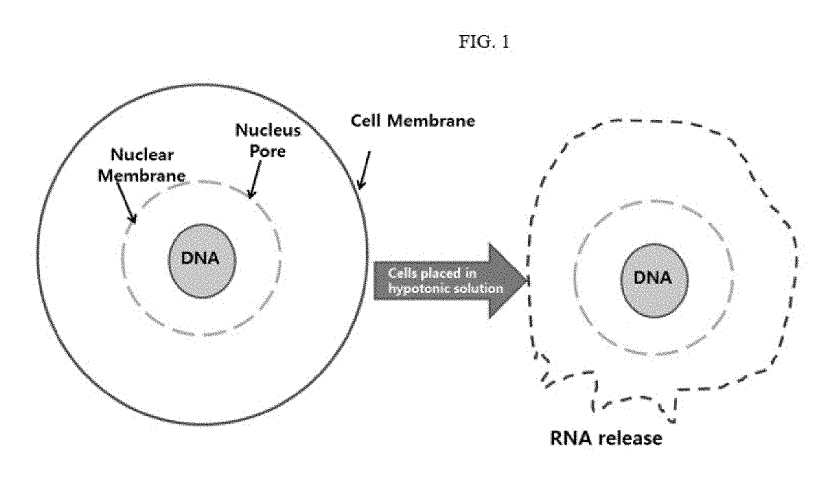

The process of cell lysis of the step (3) is schematically shown in FIG. 1. FIG. 1 shows that only the cell membrane is selectively lysed by treatment of the hypotonic solution. Since the cell membrane is lysed and eluted RNA is present in a liquid state and the nuclear membrane is maintained when the hypotonic solution is invaded, due to the structural difference of nuclear membrane having nuclear pores different from the cell membrane, and thereby DNA is present in the cell precipitate in which the complete state of nucleus is present, RNA and DNA may be independently obtained from a single cell sample.

The step of obtaining a liquid portion and a solid portion of cell lysates of the step (4) is a step of isolating a liquid portion comprising cytoplasm component of lysed cell and a solid portion comprising the complete state of nucleus in which the nuclear membrane connected to the component of cell membrane is maintained by the component of cell membrane and cytoskeleton component, and RNA eluted from the cell in the liquid portion and DNA present in the nucleus in the solid portion are comprised.

The step of obtaining a liquid portion and a solid portion of cell lysates of the step (4) may be conducted by isolating the supernatant (liquid portion) and the precipitate (solid portion) by centrifugation or magnetic separation of cell lysate obtained in the step (3). In one embodiment, in case of using a magnetic bead, the step of obtaining a liquid portion and a solid portion of cell lysates of the step (4) may be conducted by applying a magnetic field on cell lysates obtained in the step (3). For example, in case of using a magnetic bead, in the step of obtaining a liquid portion and a solid portion of cell lysates of the step (4), the solid portion (comprising DNA) is formed by applying a magnetic field generator like magnet on a container comprising the cell lysates and immobilizing the cell membrane and nucleus captured by the magnetic bead, and the free liquid portion (comprising RNA) is formed from the magnetic field.

In an embodiment of the present invention, the step of obtaining a liquid portion and a solid portion of cell lysates of the step (4) does not comprise a step of using a filter having a pore size which can filter the solid portion.

The (5) step of isolating RNA from the liquid portion and (6) step of isolating DNA from the solid portion may be carried out simultaneously or in any order.

The step of isolating RNA from the liquid portion of the step (5) may be conducted by isolating RNA from the supernatant, in case of conducting the step (4) by centrifugation, and may be conducted by isolating RNA from the liquid portion which is not immobilized on the magnetic field generator, in case of conducting the step (4) by the magnetic field generator.

As described above, in the present invention, because the targeting material of the bead targets the cell membrane of the target cell, different from conventional way of targeting mRNA using oligo-dT bound beads, all RNA present in the cell can be isolated. Thus, the isolated RNA may be one or more of all RNA kinds consisting of, for example, mRNA, rRNA, tRNA, snRNA, other non-coding RNA, and in one embodiment, may be a transcriptome comprising all of them.

The isolated RNA can be quantitatively and/or qualitatively analyzed by all common means and/or methods known in the art. Thus, after the step of isolating RNA from the liquid portion of the step (5), a step of quantitative and/or qualitative analysis of isolated RNA may be further comprised. For example, the RNA analysis may be conducted by a step of preparing cDNA by reverse transcription of RNA and a step of amplifying the obtained cDNA, by common methods. The step of amplifying cDNA may be carried out by polymerase chain reaction (PCR; for example, quantitative polymerase chain reaction (qPCR), Real-time PCR), ligase chain reaction, nucleic acid sequence-based amplification, transcription-based amplification system, strand displacement amplification, amplification through Q13 replicase, or any other appropriate methods for amplifying a nucleic acid molecule which are known in the art. In other embodiment, the RNA analysis may be carried out by common RNA analysis methods of northern blot hybridization, dot or slot blot hybridization, RNase protection assay.

The step of isolating DNA from the solid portion of the step (6) may be carried out by isolating DNA from the precipitate when step (4) is conducted by centrifugation, and may be carried out by isolating DNA from the solid portion immobilized by a magnetic field generator when the step (4) is conducted by the magnetic field generator. The DNA isolation step may comprise a step of lysing a nuclear membrane and a step of isolating eluted DNA. The step of lysing a nuclear membrane may be conducted through common methods, for example, methods of chemical lysis like alkaline lysis, detergent based lysis, or physical lysis like sonication, mechanical disruption, homogenization, freeze/thaw cycle. In case of alkaline lysis, the alkaline solution selected from the group consisting of Tris-EDTA (Ethylenediaminetetraacetic acid), sodium hydroxide/sodium dodecyl sulfate (NaOH/SDS), etc. may be used, and it may be used by adding one or more additional agents selected from the group consisting of dithiothreitol (DTT), proteinase K. randomly, but not limited thereto, and all alkaline solutions and reaction conditions which are commonly used for lysis of nuclear membrane may be properly applied. The step of isolating eluted DNA may be conducted, after the step of lysis of nuclear membrane, by isolating the supernatant obtained by centrifuging reacted materials in which the step of lysis of nuclear membrane, or by capturing and removing the nuclear membrane or isolating the solution portion through the magnetic bead, by forming magnetic field with a magnetic field generator (for example, magnet) (for example, for about 0.5 to 3 min, or about 0.5 to 2 min), when the magnetic bead is used.

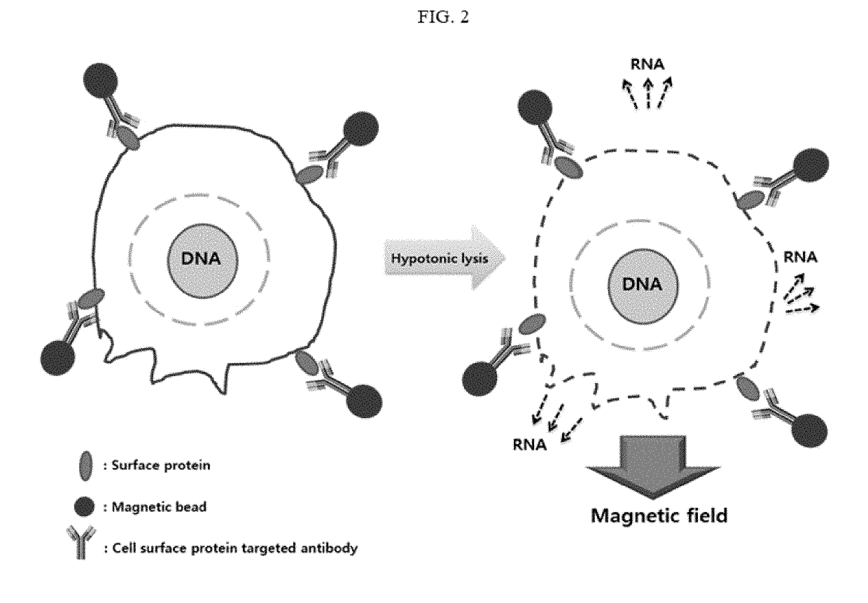

An example of the RNA and DNA isolation process is schematically shown in FIG. 2. FIG. 2 shows a process of isolating RNA and extracting (isolating) RNA from cell lysates, by selectively lysing the cell membrane using the hypotonic solution and attracting the magnetic bead connected to cell lysates by applying magnetic field, thereby isolating RNA eluted from the cell and present in the solution, after combining the magnetic bead in which the antibody specifically binding to the cell membrane surface protein and the cell.

After the step of lysing the nuclear membrane, or after the step of isolating eluted DNA, a step of removing the bead may further comprised. In case of using the magnetic bead, it may be removed by using a magnetic field generator like magnet, but not limited thereto.

The isolated DNA can be quantitatively and/or qualitatively analyzed by all common means and/or methods. Thus, after the step of isolating DNA from the solid portion of the step (6), a step of quantitative and/or qualitative analysis of isolated DNA may be further comprised. For example, the DNA analysis may be conducted by a step of amplification by common methods. The step of amplifying DNA may be carried out by polymerase chain reaction (PCR; for example, quantitative polymerase chain reaction (qPCR), Real-time PCR), multiple displacement amplification (MDA), ligase chain reaction, nucleic acid sequence-based amplification, transcription-based amplification system, strand displacement amplification, amplification through Q13 replicase, or any other appropriate methods for amplifying a nucleic acid molecule which are known in the art.

Conventional technologies have a problem in that samples for DNA/RNA analysis should be independently prepared from pooled-samples and analyzed, or DNA/RNA should be independently analyzed from an individual single cell. One embodiment of the present invention has an advantage that can simultaneously isolate, analyze and/or compare genome and transcriptome from a single cell sample, by providing a technology capable of isolating partially lysed cells using a magnetic bead and extracting DNA, after selectively lysing the cell membrane of single cell and extracting RNA.

RNA expression information of a single cell can be constructed through a whole transcriptome analysis and the expression profile distribution in the cell population can be analyzed on the basis of the individual cell information. Information available for analyzing cell individual CNV (copy number variation), through a whole genome amplification of a single cell can be provided. In addition, by analyzing genome and transcriptome simultaneously, they can be utilized for integrative SNV/InDel analysis.

In addition, the present invention provides a cDNA library which is available for a whole transcriptome analysis by effectively extracting a sub-pg level of RNA obtained from a single cell without a RNA loss. When RNA is extracted, the isolation is possible without a separate tagging or pretreatment. After extracting RNA, RNA-free DNA can be easily obtained through a magnetic separation method using a magnetic bead.

### EXAMPLES

Hereinafter, the present invention will be described in detail by examples.

However, the following examples only illustrate the present invention, but the scope of the present invention is not limited by the following examples.

### Example 1: Isolation of DNA and RNA from a Cell Sample

1.1. Fabrication of Antibody-Attached Magnetic Bead

MCF-7 cell (ATCC; Manassas, Va., ATCC® HTB-22™), which is one kind of breast cancer cell was prepared as a target cell. As a magnetic bead, Dynabeads® (Life Technologies, 10003D) with a diameter of 2.8 μm and protein G attached thereto was prepared. For capturing the component of cell membrane of the MCF-7 cell, anti-EpCAM antibody (HEA125 don; Novus, NB100-65094), which was capable of binding to EpCAM that was one of cell membrane proteins of MCF-7 cell, was prepared.

A magnetic bead solution was prepared by mixing the prepared magnetic bead with PBS (pH7.4) containing 0.1% (w/v) BSA (bovine serum albumin). After mixing 100 μl of the prepared magnetic bead solution and 700 μl of anti-EpCAM antibody undiluted solution and reacting them at 4 □ overnight, the unreacted material was removed by collecting the magnetic bead for 1 min using a magnet and removing supernatant. 200 μl of washing buffer (PBS comprising 0.1% BSA containing 2 mM EDTA, pH7.4) was added herein, and the step of washing was repeated three times. 100 μl of buffer solution (PBS comprising 0.1% BSA, pH 7.4) was added to the obtained reacted materials, and a magnetic bead solution at which anti-EpCAM antibody was attached was prepared.

1.2.1. Bond of Target Cell and Antibody-Bound Magnetic Bead

100 μl PBS solution (pH7.4) comprising approximately 5*10³ number of MCF-7 cells prepared above was put in a tube, and the antibody-attached magnetic bead solution prepared in the Example 1.1 was added herein in an amount containing about 5*10⁴ number of magnetic beads. By reacting for approximately 10 to 20 min at a room temperature with stirring, the antibody-attached magnetic beads and cells were bound. After the tube comprising cells and magnetic beads was positioned on the magnet for 1 min, unbound cells were removed by removing supernatant. 100 μl buffer solution (PBS, pH7.4) was added herein, thereby preparing a solution of cell bound with magnetic beads.

1.2.2. Single Cell Calculation and Reconfirmation

The prepared MCF-7 cells at which beads were bound were diluted at the concentration of single cell/1 μl using the buffer solution (PBS, pH7.4). 1 μl of diluted cell solution was pipetted on 5 wells of 96 well plate. It was confirmed whether a single cell was dispensed on each well using a microscope. 1 μl of solution was pipetted from the solution of which a single cell/1 μl level of concentration was confirmed (master solution), thereby dispensing it on 10 wells, and then it was reconfirmed whether a single cell was present in each well with the microscope.

1.3. Cell Membrane Lysis by Hypotonic Solution

A hypotonic solution was added to the single cell, which magnetic beads were bound at, prepared in the Example 1.2.2, thereby lysing cells.

Specifically, the hypotonic solution was prepared by adding RNase inhibitor (Clontech, 070814) in an amount of 1% (v/v) to the solution in which Triton X-100 was dissolved in a concentration of 0.1% (v/v) in the buffer aqueous solution containing water (distilled water) and PBS (pH 7.4) at 4:1 (v:v). The prepared hypotonic solution 9 μl was added to 1 μl of solution of cell, which magnetic beads were bound at, prepared in the Example 1.2.2 (PBS solution comprising 1 cell) and it was reacted for 10 min at a room temperature, thereby obtaining cell lysates in which cell membrane was lysed.

1.4. RNA and DNA Extraction

A magnet was positioned on a reactor comprising cell lysates obtained in the Example 1.3 and cell lysates containing DNA were attracted with the magnet, and the solution comprising eluted RNA was isolated by using a pipet, thereby extracting RNA.

In addition, after lysis of nuclear membrane using alkaline lysis method, DNA was eluted. Specifically, PBS (pH7.4) 4 μl was added to cell lysates remained after isolating the solution comprising RNA. After alkaline lysis buffer (1M DTT 3 μl, Buffer DLB 33 μl; Qiagen, 150343) was added in an amount of 3 μl and reacted for 10 min at 65□, the reaction was finished by adding stop solution 3 μl, thereby lysing nuclear membrane. In order to prevent DNA loss in the process of removing beads, the whole genome DNA analysis was carried out with the beads attached.

### Example 2: Confirmation of Selective Lysis by Hypotonic Solution

In order to confirm that the nuclear membrane was maintained but only the cell membrane was selectively lysed by the hypotonic lysis which uses a hypotonic solution, the following experiment was carried out.

The cytoplasm and nucleus of MCF-7 cell were stained with CellTracker™ green CMFDA (Life Technologies; cytoplasm) and 4′,6-diamidino-2-phenylindole (DAPI, blue; nucleus), respectively. Then, the isotonic solution (PBS; pH 7.4) and hypotonic solution (for lysis of the ratio of PBS (pH 7.4):water=1:5 (v;v)) were added to cell solution 100 μl (comprising about 5*10⁴ cells) in an amount of 500 μl, and reacted for 10 min at a room temperature.

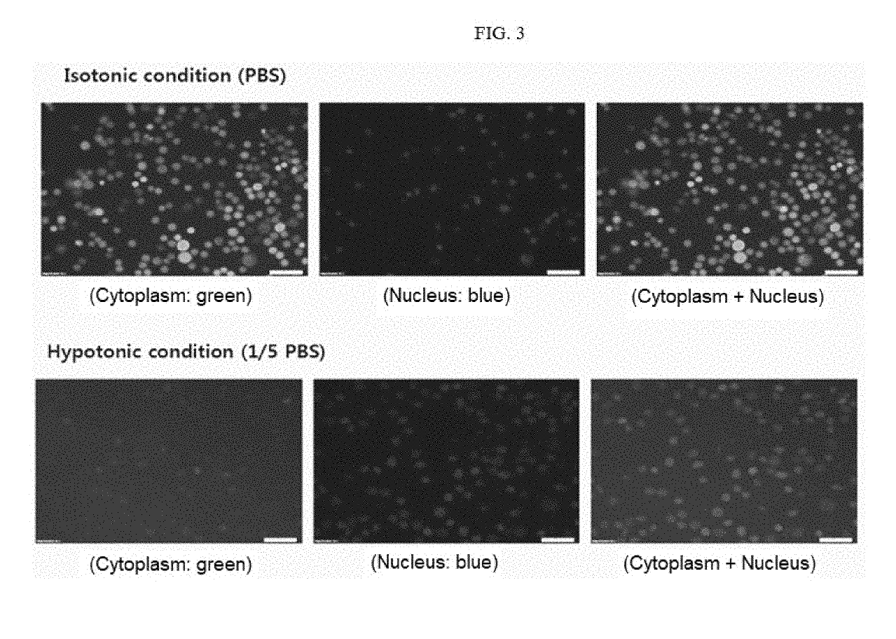

The fluorescence image obtained by observing reacted cells as above using the fluorescence microscope (Olympus, IX81-XDC) is shown in FIG. 3. As confirmed in FIG. 3, while the cytoplasm (green) and nucleus (blue) were completely preserved in case of treating the isotonic solution (PBS), in case of treating the hypotonic solution (⅕ PBS), the nucleus (blue) was completely preserved but the cytoplasm (green) was dissolved and spread in the solution.

### Example 3: Quantification Analysis of RNA and DNA

The method of isolating a nucleic acid disclosed in the Example 1 was applied to 10 MCF7 cells, thereby isolating DNA and RNA. By qualifying DNA and RNA isolated like so, they were compared with DNA and RNA comprised in the whole cell lysate (Intact cell).

Quantification of DNA was conducted by progressing quantitative real-time PCR (qRT-PCR); Light Cycler 480 II (Roche)) using line 1 locus as a target, and the amount of DNA was relatively shown by using obtained Cp values. The primers and PCR conditions used in the qRT-PCR were as follows:

1) Primers,

hLINE1 Forward:
(SEQ ID NO 1)
TCA CTC AAA GCC GCT CAA CTA C
hLINE1 Reverse:
(SEQ ID NO 2)
TCT GCC TTC ATT TCG TTA TGT ACC

2) PCR Conditions

Components: SYBR Green master mix (Exiqon, 203400) 10 μl, isolated DNA diluted 1:2 with 1× TE Buffer, pH 8.0, 5 μl, 10 uM forward and reverse primer 0.2 μl each, Nuclease-free water 4.6 μl,

Reaction condition: Holding Enzyme activation 95□ 10 min, Cycling (40 cycles) Denature 95□ 10 sec, Anneal/extend 60□ 1 min.

After fabricating cDNA library using GAPDH as a target, RNA was qualified by using Cp values obtained by progressing TaqMan assay. Specifically, the fabrication of cDNA library was progressed by using the reagent of Single Cell-to-CT™ Kit (Life Technologies, 4458237). After adding DNase I 1 μl to the 10 μl solution comprising RNA, reverse transcription was implemented (Components: Single Cell VILO™ RT Mix 3.0 Single Cell SuperScript® RT 1.5 μl; reaction condition: 25□ 10 min, 42□ 60 min, 85□ 5 min).

cDNA synthesized as above was pre-amplified under the following conditions:

components: Single Cell PreAmp Mix 5 μl, 0.2× pooled TaqMan® Gene Expression Assays 6 μl) Total PreAmp reaction mix 11 μl; reaction condition: Holding Enzyme activation 95□ 10 min, Cycling (14 cycles) Denature 95□ 15 sec, Anneal/extend 60□ 4 min, Holding Enzyme Deactivation 99□ 10 min)

TaqMan assay was carried out by using Light Cycler 480 II (Roche):

Primer: Hs03929097_g1 (Life Technologies),

Reaction Conditions:

Components: 2× TaqMan® Gene Expression Master Mix 10 μl, Preamplified product diluted 1:20 with 1× TE Buffer, pH 8.0, 4 μl, 20× TaqMan® Gene Expression Assay 1 μl, Nuclease-free water 5 μl,

Reaction condition: Holding UDG incubation 50□ 2 min, Holding Enzyme activation 95□ 10 min, Cycling (40 cycles) Denature 95□ 5 sec, Anneal/extend 60□ 1 min

Cp value measurement: Light Cycler 480 II (Roche).

Cp (Crossing point) value means the cycle number at which a detectable fluorescence signal appears in a real-time PCR reaction. In other words, the higher the primary DNA concentration is, detection of fluorescence signal is possible in the lower Cp value, and the lower the primary DNA concentration is, detection of fluorescence signal is possible in the higher Cp value. That is, quantification of DNA is possible through comparison of Cp values.

For comparison, DNA and RNA comprised in the whole cell lysate (Intact cell) were quantified (real-time PCR) by the aforementioned method (progressed without a quantification process before reaction under the premise in that the similar level of DNA/RNA was present, when the equal number of cells were inserted).

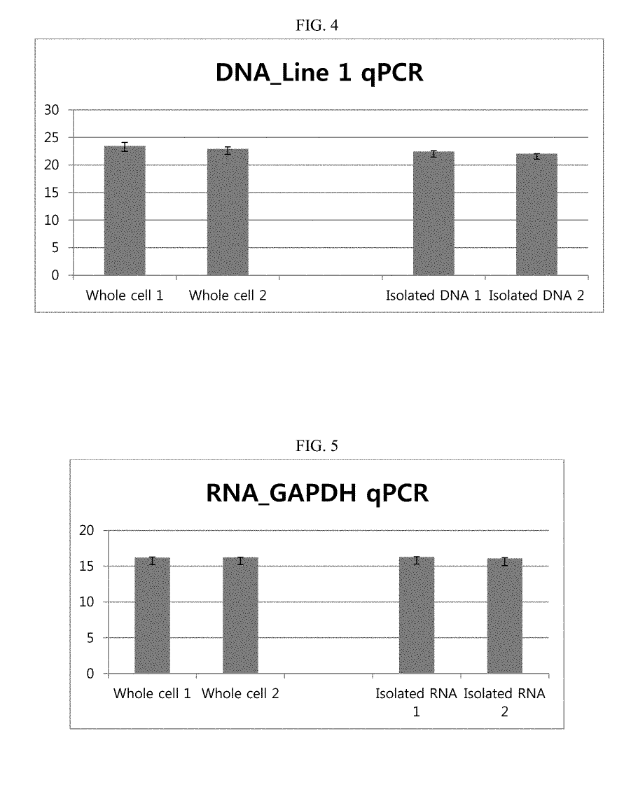

The DNA and RNA quantification was progressed in 2 wells each, and the obtained Cp values were shown in the following Table 1 (result of DNA quantification) and Table 2 (result of RNA quantification), and among them, the values obtained from 10 cell analyses were shown in FIG. 4 (result of DNA quantification) and FIG. 5 (result of RNA quantification).

TABLE 1
Line 1 qRT-PCR

| | 10 cell | |
|---|---|---|
| Line 1 | 1 | 2 |
| Intact cells | 23.9 | 23.17 |
| | 23.01 | 22.66 |
| Isolated DNA | 22.54 | 22.05 |
| | 22.32 | 22.05 |

Data: Cp value

TABLE 2
GAPDH gene expression TaqMan assay

| | 10 cells | |
|---|---|---|
| GAPDH | 1 | 2 |
| Intact cells | 16.26 | 16.25 |
| | 16.16 | 16.23 |
| Isolated RNA | 16.26 | 16.15 |
| | 16.32 | 16.01 |

Data: Cp value

As shown in the Tables 1 and 2, and FIGS. 4 and 5, it was confirmed that the relative amount of isolated DNA and RNA was in the similar level with the whole cell lysate, and this verified that DNA and RNA were extracted (isolated) without a loss, and in addition, the result in 10 cells was similarly obtained, showing that the method of isolating according to Example 1 could be effectively applicable for even the case that the number of cells was few.

### Example 4: Recovery Rate Test of RNA and DNA (DNA/RNA Recovery Rate Validation)

4.1. RNA Recovery Rate Test

Three kinds of RNA samples, such as RNA extracted from 10 MCF7 cells (Intact cells; After RNA recovery by the method which skips a step of isolating a portion of cell membrane attached to beads using a magnet in Example 1, cDNA was synthesized), RNA fraction in which the portion of cell membrane comprising DNA was isolated from 10 MCF7 cells using the method of isolating a nucleic acid of Example 1 (Isolated RNA), RNA lost as DNA fraction by being adsorbed to magnetic beads during RNA isolation from 10 MCF7 cells using the method of isolating a nucleic acid of Example 1 (Residual RNA; After removing supernatant comprising RNA and then additionally inserting lysis solution 10 μl to the solid portion in which beads and cell membrane were bound in order to analyze RNA adsorbed on beads, the process of synthesizing cDNA was progressed), were quantitatively analyzed by performing TaqMan assay by fabricating cDNA library using GAPDH as a target.

(Primer:

Taqman gene expression analysis (Life Technologies, Hs03929097_g1),

Reaction Conditions:

Components: 2× TaqMan® Gene Expression Master Mix 10 μl, Preamplified product diluted 1:20 with 1× TE Buffer, pH 8.0, 4 μl, 20× TaqMan® Gene Expression Assay 1 μl, Nuclease-free water 5 μl,

Reaction condition: Holding UDG incubation 50□ 2 min, Holding Enzyme activation 95□ 10 min, Cycling (40 cycles) Denature 95□ 5 sec, Anneal/extend 60□ 1 min,

Cp values: measuring with Light Cycler 480 II (Roche).

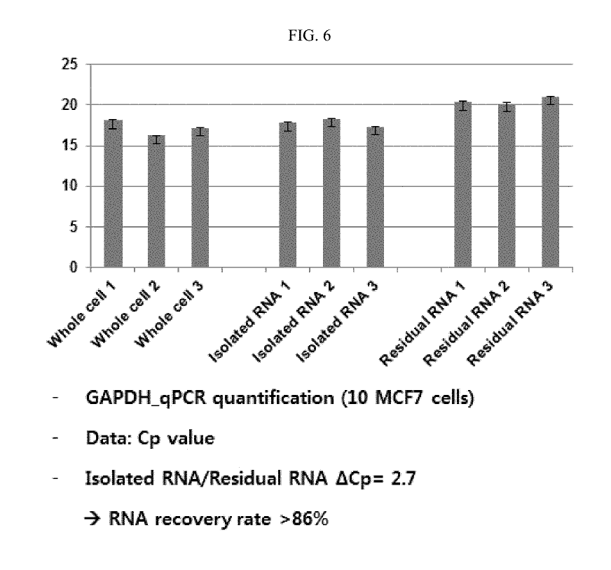

The quantification process was carried out three times each, and the obtained Cp values were shown in FIG. 6, and the Cp average value was shown in the following Table 3.

TABLE 3
GAPDH gene expression TaqMan assay

| | Intact cells | Isolated RNA | Residual RNA |
|---|---|---|---|
| Average Cp | 17.17 | 17.81 | 20.52 |

Data: Cp value

As shown in the Table 3 and FIG. 6, isolated RNA which was isolated according to Example 1 exhibited a similar level of Cp value with the intact cell RNA (RNA derived from whole cell). In other words, the difference of the average Cp values of isolated RNA and residual RNA (ΔCp) was 2.71, and the fold change of two values of RNA amounts exhibited the ratio of Isolated RNA:residual RNA=2^{2.71}:1=6.54:1, and thus it was shown that the amount of isolated RNA compared with residual RNA accounted for approximately 86% of the total, and the amount of residual RNA compared with isolated RNA accounted for approximately 14% of the total. Such result exhibited that RNA could be effectively extracted from a few number of cells through the method of isolating a nucleic acid according to Example 1, and it shows the applicability to a single cell of the technology of such Example 1.

4.2. DNA Recovery Rate Test

Three kinds of DNA samples, such as DNA extracted from 10 MCF7 cells (Intact cells; n=3; After implementing lysis by the method which skips a step of isolating a portion of cell membrane attached to beads using a magnet in Example 1, DNA was recovered), DNA fraction in which RNA was isolated from 10 MCF7 cells using the method of isolating a nucleic acid of Example 1 (Isolated RNA; n=3), DNA lost during RNA isolation from 10 MCF7 cells using the method of isolating a nucleic acid of Example 1 (Residual RNA; n=3; for confirming DNA released to the liquid portion comprising RNA fraction due to selective lysis of nuclear membrane in the process of cell membrane lysis, lost DNA which was not bound to the magnet with cell membrane by binding to magnetic beads and was contained in the liquid portion (lysis buffer) was quantified through real time PCR), etc, were quantitatively analyzed by using qRT-PCR.

For DNA, real-time PCR was progressed by using line 1 locus as a target, and the amount of DNA was relatively compared by using Cp values (refer to Example 3).

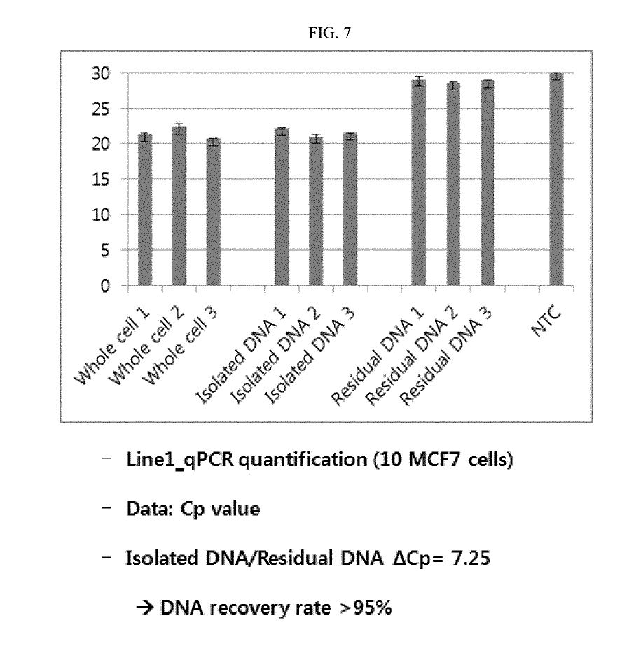

The quantification process was carried out twice each for all samples, and the average of obtained Cp values of all samples was shown in FIG. 7 (NTC was the Cp value of the negative control in which DW was added instead of DNA sample), and the Cp average value per DNA (Intact cell, Isolated DNA, Residual DNA) was shown in the following Table 4.

TABLE 4
Line 1 qRT-PCR

| | Intact cells | Isolated DNA | Residual DNA |
|---|---|---|---|
| Average Cp | 21.46 | 21.56 | 28.82 |

Data: Cp value

As shown in the Table 4 and FIG. 7, the difference of the average Cp values of isolated DNA which was isolated according to Example 1 and residual DNA, ΔCp was 7.26, and the fold change of two values of DNA amounts exhibited the ratio of 2^{7.26}:1=153.5:1, and thus it was shown that the amount of isolated DNA compared with residual DNA accounted for approximately 99% of the total, and the amount of residual DNA compared with isolated DNA accounted for approximately 1% of the total. Such result exhibited that DNA could be effectively extracted from a few number of cells through the method of isolating a nucleic acid according to Example 1, and it shows the applicability to a single cell of the technology of such Example 1.

### Example 5: Sequence Correlation Analysis

The correlation analysis between RNA sequence of MCF7 bulk sample (MCF7_B) (1*10⁶ cell or more used; 1 ng of cDNA obtained from the cell was used for RNA-sequencing, RNA sequence of the whole cell of MCF7 single cell, and the sequence of RNA isolated by using the method of isolating a nucleic acid of Example 1 from MCF7 single cell (fractionated or isolated RNA; RNA isolated by the method of isolating a nucleic acid of Example 1) was performed.

Specifically, one kind of MCF7 bulk sample, 10 kinds of whole RNA samples, and 10 kinds of fractionated RNA samples were objects for analysis, and the average value of whole/fractionated sample gene expression level was earned. The gene expression level was conducted by the RNA quantification analysis method as aforementioned in the Example 3.

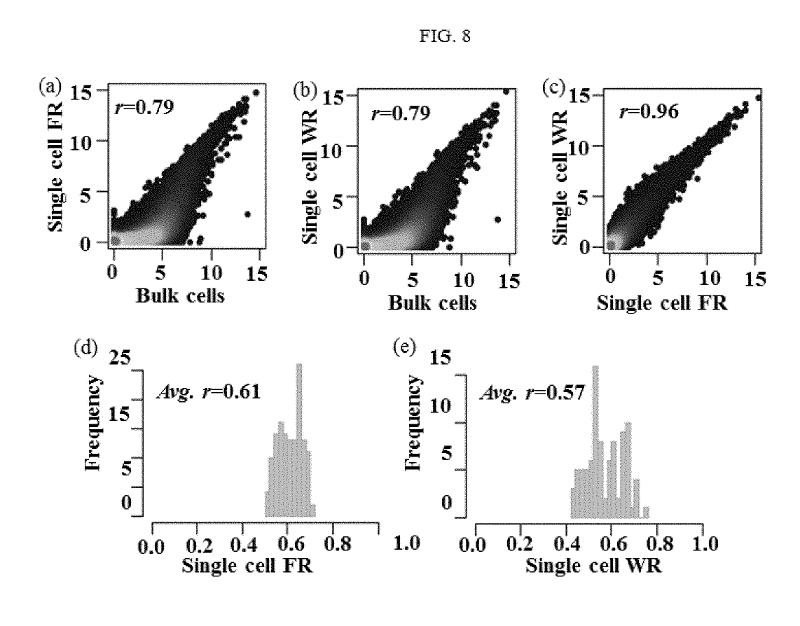

The result of correlation of the obtained gene expression profile was shown in (a) to (c) of FIG. 8. In (a) to (c) of FIG. 8, the average value of gene expression level obtained from MCF7 bulk sample (marked as “Bulk cells”) vs. gene expression level obtained from whole RNA samples (marked as “Single cell WR”), the average value of gene expression level obtained from MCF7 bulk sample vs. gene expression level obtained from fractionated RNA samples (marked as “Single cell FR”), and the average value of gene expression level obtained from whole RNA samples vs. gene expression level obtained from fractionated RNA samples were schematized as a scattered plot, respectively, thereby showing correlation of expression levels between samples. Herein r represents a correlation coefficient (the correlation coefficient represents the level of sequence data similarity and/or correlation level). The numerical values of x axis and y axis of each graph represent gene expression level, namely RNA level.

(d) and (e) of FIG. 8 are graphs showing the cell-to-cell correlation coefficient in each parent population of single cell fraction/single cell whole, and frequency of y axis means the number of pair having the corresponding correlation coefficient value. RNA expression correlation between about ˜10 cells of FR (single cell fraction) and WR (single cell whole) was analyzed, and each average correlation coefficient was r=0.61 (single cell FR), and r=0.57 (single cell WR), respectively, and therefore it was confirmed that there was no significant difference between them.

As shown in FIG. 8, the result of analysis of RNA isolated by the method of isolating a nucleic acid of Example 1 had equal or higher isolation efficiency to the conventional method of analyzing RNA derived from the whole cell.

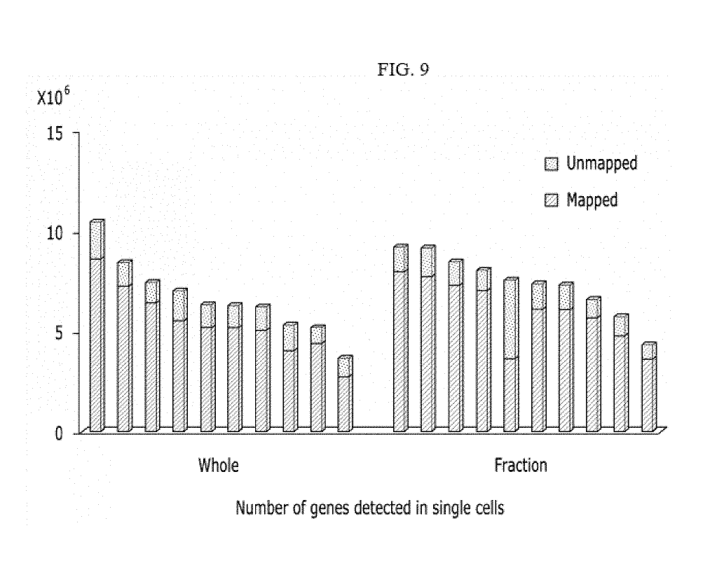

In addition, the detected gene number was shown in FIG. 9 as the result of RNA sequencing of RNA samples isolated by using the method of isolating a nucleic acid of Example 1 from the obtained MCF7 single cell derived whole cell and MCF7 single cell (fractionated RNA sample). In FIG. 9, detect gene represents the number of genes detected as the result of sequencing, and unmapped represents the number of genes not mapped in the reference sequence, and mapped represents the number of genes mapped in the reference sequence (human genome reference: hg19 (UCSC genome browser); method of analysis: calculating the number of reads mapped or unmapped in the reference among the total read counts by mapping hg19 sequence as a reference and sequence read of sequencing-completed sample).

As shown in FIG. 9, there was no large difference in detected gene numbers of MCF7 single cell derived whole cell and fractionated RNA sample. On the basis of that, it was demonstrated that the method of analysis of the present invention exhibited the equal level compared to the conventional method (method for independently analyzing RNA without separating DNA/RNA).

### Example 6: Whole Genome Sequencing (WGS)

The performance evaluation to the single cell whole genome amplification of the method of isolating a nucleic acid of Example 1 by using MCF7 cells was carried out. Whole genome sequencing (WGS) was conducted to MCF7 bulk sample (1*10⁶ cell or more used; conducting WGS to gDNA obtained in the cells), DNA fractions obtained from MCF7 single cells by the method of the Example 1, and genome DNA (gDNA) obtained from whole cell lysates of MCF7 single cells, thereby measuring genome wide copy number variations.

DNA library for whole genome sequencing was prepared by using TruSeq Nano DNA Library Prep Kit (Illumina, USA), and analysis was progressed with 100 bp paired-end mode by using Illumina HiSeq 2500. Read depth was progressed as 0.1× to 0.7×, and all sequencing reads were aligned to Hg 19 reference genome by using BWA aligner (bio-bwa.sourceforge.net).

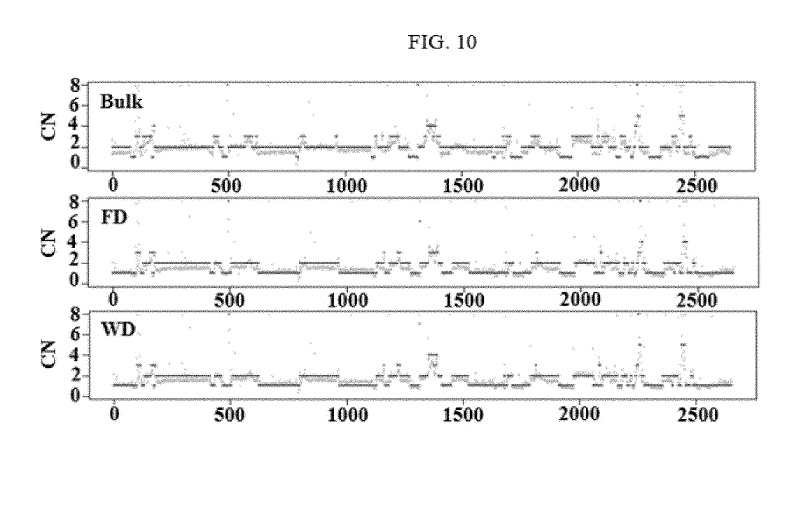

The obtained result was shown in FIG. 10. In FIG. 10, −CN of Y axis represents copy number, and Bulk is the WGS result of MCF7 bulk sample (copy number), and FD is the copy number of DNA fractions obtained from MCF7 single cells by the method of isolating a nucleic acid of Example 1, and WD represents the copy number of gDNA obtained from whole cell lysates of MCF7 single cells. The numerical value of X axis of each graph means chromosome region. As shown in FIG. 10, it was demonstrated that the copy number profile obtained from DNA fractions obtained from MCF7 single cells by the method of isolating a nucleic acid of Example 1 had no large difference from the copy number profile obtained from gDNA obtained from whole cell lysates of MCF7 single cells and MCF7 bulk samples. Such result means that the result of analyzing a nucleic acid for the nucleic acid isolated from a single cell by the method of isolating a nucleic acid illustrated in Example 1 in the similar level with bulk sample or whole cell could be obtained

1. A method of isolating a nucleic acid from a single cell, comprising
(1) providing a cell sample comprising a target cell;
(2) treating the cell sample with a bead to which a targeting material is attached, wherein the targeting material binds to a protein on the membrane of the target cell, and binds the bead to the membrane of the target cell;
(3) treating the cell sample with a hypotonic solution to provide a cell lysate;
(4) obtaining a liquid portion and a solid portion of the obtained cell lysate;
(5) isolating RNA from the liquid portion obtained from the step (4); and
(6) isolating DNA from the solid portion obtained from the step (4).

2. The method of isolating a nucleic acid of claim 1, wherein the bead is selected from the group consisting of a magnetic bead, a silica bead, a polymer bead, a glass bead, a cellulose bead, a quantum dot (Q-dot), and a metal bead.

3. The method of isolating a nucleic acid of claim 1, wherein the targeting material is at least one selected from the group consisting of an antibody, an antigen binding fragment of an antibody, a protein scaffold, and an aptamer, which binds to the protein on the cell membrane surface.

4. The method of isolating a nucleic acid of claim 1, wherein the targeting material is attached to the surface of bead by a ligand-receptor bond, an ionic bond, a covalent bond, or adsorption.

5. The method of isolating a nucleic acid of claim 1, wherein the hypotonic solution is a) a buffer solution, or b) a surfactant solution comprising a surfactant dissolved in water or a buffer solution.

6. The method of isolating a nucleic acid of claim 5, wherein the buffer solution of a) or b) comprises phosphate buffer saline (PBS), Hank's balanced saline solution (HBSS), or a mixture thereof, in water at a volume ratio of 90:10 to 70:30 (water volume:buffer volume).

7. The method of isolating a nucleic acid of claim 6, wherein the buffer solution of a) or b) comprises phosphate buffer saline (PBS), Hank's balanced saline solution (HBSS), or a mixture thereof, in water at a volume ratio of 85:15 to 75:25 (water volume:buffer volume).

8. The method of isolating a nucleic acid of claim 6, wherein the buffer solution of a) or b) comprises phosphate buffer saline (PBS), Hank's balanced saline solution (HBSS), or a mixture thereof, in water at a volume ratio of 82:18 to 78:22 (water volume:buffer volume).

9. The method of isolating a nucleic acid of claim 5, wherein the surfactant is at least one selected from the group consisting of polyoxyethylene octylphenylether, polysorbate, and 3-[(3-Cholamidopropyl)dimethylammonio]-1-propanesulfonate.

10. The method of isolating a nucleic acid of claim 5, wherein the concentration of the surfactant in the surfactant solution is 0.05 to 0.5% (v/v) based on the volume of water or buffer solution.

11. The method of isolating a nucleic acid of claim 10, wherein the concentration of the surfactant in the surfactant solution is 0.1 to 0.3% (v/v) based on the volume of water or buffer solution.

12. The method of isolating a nucleic acid of claim 1, wherein the step of obtaining a liquid portion and a solid portion of the cell lysate is performed by centrifuging the cell lysate obtained from step (3).

13. The method of isolating a nucleic acid of claim 1, wherein the bead is a magnetic bead, and the step of obtaining a liquid portion and a solid portion of the cell lysate is performed by applying a magnetic field to the cell lysate obtained from step (3).

14. The method of isolating a nucleic acid of claim 1, wherein the cell sample contains a single cell.

15. (canceled)

16. The method of isolating a nucleic acid of claim 1, wherein the step (4) does not comprise a step of using a filter.
